(19) United States
(12) Patent Application Publication
(10) Pub. No.: US 2010/0160358 A1
(43) Pub. Date: Jun. 24, 2010

(54) Pyridylsulfonamidyl-Pyrimidines for the Prevention of Blood Vessel Graft Failure

(76) Inventors: Christoph Schumacher, Allschwil (CH); Jessica Mann, Basel (CH)

Correspondence Address: WENDEROTH, LIND & PONACK, L.L.P., 1030 15th Street, N.W.,, Suite 400 East, Washington, DC, 20005-1503, US

(21) Appl. No.: 11/991,782

(22) PCT Filed: Sep. 11, 2006
(86) PCT No.: PCT/EP2006/066250

§ 371 Date: Mar. 11, 2008

(30) Foreign Application Priority Data
Sep. 12, 2005 (EP) 05108340.0

(51) Int. Cl. A61K 31/506; C07D 239/10; A61P 9/10

(52) U.S. Cl. 514/269; 544/298

(57) ABSTRACT

The present invention relates to the use of a compound of formula (I), wherein R₁ is pyridyl or thiazolyl, any of which may optionally be substituted with C₁₋₈alkyl or C₂₋₈alkenyl; and a) R₂ is methoxy and n is zero or one; or b) R₂ is chlorine and n is zero; and pharmaceutically acceptable salts thereof for the prevention of blood vessel graft failure in patients undergoing artery bypass graft surgery.

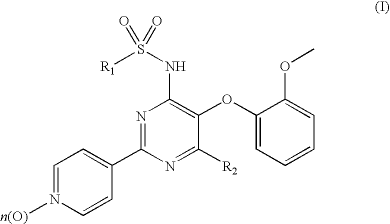

### FIELD OF THE INVENTION

The present invention relates to a new medicament/method for the prevention of blood vessel graft failure in patients undergoing artery bypass grafting comprising the use of specific pyridylsulfonamido pyrimidines.

### BACKGROUND OF THE INVENTION

The development and implementation of artery bypass graft surgery has both relieved symptoms and improved survival in patients with symptomatic and asymptomatic atherosclerosis, a disease that is the leading cause of death in the Western world. The indications for an operation have been expanded to a diversity of clinical syndromes and anatomic subsets of patients with ischemic heart disease and/or peripheral arterial occlusive disease. These include patients with stable and unstable angina pectoris, patients with acute myocardial infarction, patients with silent ischemia, survivors of sudden cardiac death, patients with congenital coronary abnormalities and patients who present with congestive heart failure secondary to reversible ischemia. The major clinical benefit of coronary artery bypass graft surgery is related to the relief of ischemia and the prevention of subsequent myocardial events. Surgical bypass of peripheral arterial occlusive disease provides an effective means to restoring blood flow to the lower extremity and has been a standard therapy for patients with disabled claudication or critical limb ischemia. Therefore, early and late bypass graft patency and limiting progression of disease in both the native coronary and peripheral circulation, respectively, and the bypass conduit are paramount.

The greater autologous saphenous vein is, despite supported advantages for using arterial grafts still the most commonly used coronary or infrainguinal bypass conduit and is particularly effective in patients with multivessel disease and diabetes. Unfortunately, the long-term results of artery bypass graft surgery are limited by stenosis and subsequent occlusion grafted vessels resulting in failure rates of 20% and 50% at 5 years and 10 years respectively (Campeau et al., Circulation, 1983, volume 68, page II 1-7; Vaislic et al., Union Med Can, 1983, volume 112, pages 229-234, Whittemore and Belkin, Vasc. Surgery, volume 1, pages 794-814). Vein or artery graft failure can be treated with repeat operation or percutaneous revascularization. However, repeat operation is associated with high mortality and morbidity. Also, percutaneous treatment of vessel graft disease is complicated by a high rate of procedural and long term complications due to the interrelated phenomena of distal embolization, slow flow or no reflow, periprocedure myocardial infarction and subsequent re-stenosis. Therefore, the prevention of graft stenosis rather than treatment of an established lesion would make a significant impact on long-term patency and, in view of the large numbers of patients receiving venous or artery bypass grafts, the development of preventative therapeutic approaches is an important aim.

Following coronary artery or peripheral bypass graft surgery, the grafted vessel is exposed to increased blood flow and pressure in the arterial system. The resulting alterations in shear and wall stress as well as endothelial injury as a consequence of bypass grafting are thought to contribute to subsequent vasculopathy that leads to intimal hyperplasia (neointima). Vein or artery graft thickening is determined by increased medial thickening and neotinima formation. The migration and proliferation of smooth muscle cells in response to a host of released growth factors and cytokines, including platelet-derived growth factor, thrombin and endothelin-1, play key roles in the development of intimal hyperplasia. Neointimal hyperplasia can lead to lumen compromise, blood flow reduction and subsequent graft failure.

### DETAILED DESCRIPTION OF THE INVENTION

The present invention relates to compounds of formula (I)

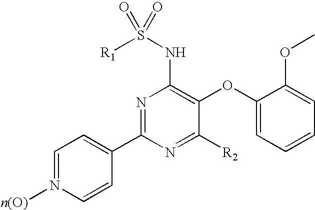

wherein
R₁ is pyridyl or thiazolyl, any of which may optionally be substituted with C₁₋₈alkyl or C₂₋₈alkenyl; and
a) R₂ is methoxy and n is zero or one; or
b) R₂ is chlorine and n is zero
and pharmaceutically acceptable salts thereof.

The present invention specifically relates to the use of a compound of formula (I) for the manufacture of a medicament for preventing blood vessel graft failure after artery bypass grafting in mammals, especially in humans. The bypass may occur with blood vessels of either venous or arterial phenotype such as the saphenous or cubital vein and the internal mammary (thoracic) or the gastroepiploic artery, respectively. Preferred blood vessels are of autologous nature. The vessel may be implanted to a coronary artery for a coronary artery bypass graft (CABG) or a peripheral artery like the femoral artery for femoropopliteal, femorocrural bypass graft or infra-inguinal bypass surgery (IIBS).

Furthermore, the present invention relates to a method for the prevention of late blood vessel graft failure after artery bypass graft surgery, that comprises the administration of an therapeutically effective amount of a compound of formula (I) to a, preferably, human subject or a mammalian animal.

The term “prevention” as used throughout the description of the present invention is meant to include also “treatment” and “delay of progression”. In particular, the term “prevention” comprises the prevention of vessel stenosis or the prolongation of vessel patency and thus the reduction of graft failures, the reduction of necessary pharmacological or surgical interventions and the reduction of mortality rates.

The sulfonamides of the present invention are known as inhibitors of endothelin receptors and a method of preparation is disclosed in WO 00/52007.

More particularly, the present invention relates to the following compounds of formula (I): R₁ is preferably 2-pyridyl or 2-thiazolyl, each optionally substituted with C₁₋₈alkyl or C₂₋₈alkenyl, and most preferably 2-pyridyl, optionally substituted with C₁₋₈alkyl or C₂₋₈alkenyl, C₁₋₈alkyl or C₂₋₈alkenyl are branched or straight chain radicals, for example methyl, ethyl, n-propyl, isopropyl, n-butyl, isobutyl, t-butyl, vinyl, 1-propenyl, allyl, isopropenyl, 1-butenyl, 2-butenyl, 3-butenyl, and the like. Preferred are said radicals which have up to (and including) four carbon atoms. Most preferred substitution is by a methyl group.

Particularly preferred are compounds of formula (I) wherein R₁ is 2-pyridyl optionally substituted with C₁₋₄alkyl; and R₂ is methoxy and n is zero and pharmaceutically acceptable salts thereof.

Most preferred is 5-methyl-pyridine-2-sulfonic acid{6-methoxy-5-(2-methoxy-phenoxy)-2-pyridin-4-yl-pyrimidin-4-yl}-amide.

The term “pharmaceutically acceptable salts” comprises salts of the compounds of formula (I) with inorganic or organic acids such as hydrochloric acid, hydrobromic acid, nitric acid, sulphuric acid, phosphoric acid, citric acid, formic acid, maleic acid, acetic acid, succinic acid, tartaric acid, methansulphonic acid, p-toluenesulphonic acid and the likes, which are non-toxic to mammals. It also includes salts with inorganic or organic bases such as alkali salts like sodium and potassium salts, alkaline earth metal salts like calcium and magnesium salts, N-methyl-D-glutamine salts and salts with amino acids like arginine, lysine and the like.

It will be appreciated that the compounds of formula (I) of this invention may be derivatized at functional groups to provide prodrug derivatives that are capable in vivo of converting back to the parent compounds. Additionally, any physiologically acceptable equivalents of the compounds of general formula (I), which are capable of producing the parent compounds of general formula (I) in vivo, are within the scope of this invention.

As mentioned above, the use of the compound of formula (I) for the manufacture of a medicament for the prevention of blood vessel graft failure after artery bypass graft surgery is an object of the instant invention, which manufacture comprises bringing one or more compounds of formula (I) and, if desired, one or more other therapeutically valuable substances into a pharmaceutical administration form.

The pharmaceutical compositions may be administered orally, for example in form of tablets, coated tablets, sugar-coated pills, hard or soft gelatine capsules, solutions, emulsions or suspensions. Administration can also occur rectally, for example by using suppositories; locally or percutaneously, for example by using ointments, creams, gels, solutions or compound-coated intravascular stents as well as extravascular cuffs; or parenterally e.g. intravenously, intramuscularly, subcutaneously, intrathecally or transdermally by using for example injectable solutions. Furthermore, administration can occur as sublingual or opthalmological preparation or as an aerosol, for example in the form of a spray.

For the preparation of tablets, coated tablets, sugar-coated pills or hard gelatine capsules, the compound of the present invention may be mixed with pharmaceutically inert, inorganic or organic excipients. Examples of suitable excipients for tablets, sugar-coated pills or hard gelatine capsules include lactose, corn starch or derivatives thereof, talc and stearic acid or salts thereof.

Suitable excipients for use with soft gelatine capsules may include for example vegetable oils, waxes, fats, semi-solid or liquid polyols etc.

Useful excipients for the preparation of solutions and syrups may include for example water, polyols, saccharose, invert sugar and glucose.

Useful excipients for the preparation of injectable solutions may include for example water, alcohols, polyols, glycerine and vegetable oils.

Useful excipients for the preparation of suppositories and other local or percutaneous applications may include, for example natural or hardened oils, waxes, fats and semi-solid or liquid polyols.

The following examples illustrate possible administration forms:

Tablets containing the following ingredients can be produced in a conventional manner:

| Ingredients | mg per tablet |
|---|---|
| Compound of formula (I) | 0.1-500 |
| Lactose | 125 |
| Corn starch | 75 |
| Talc | 4 |
| Magnesium stearate | 1 |

Capsules containing the following ingredients can be produced in a conventional manner:

| Ingredients | mg per capsule |
|---|---|
| Compound of formula (I) | 0.1-500 |
| Lactose | 150 |
| Corn starch | 20 |
| Talc | 5 |

Injection solutions may have the following composition:

| Ingredients | Amount |
|---|---|
| Compound of formula (I) | 0.01-50 |
| Sodium chloride | 8.5 |
| Tris(hydroxymethyl)aminoethane | 0.5 |
| HCl 0.1 N | Ad pH 8.0 |
| Water for injection | Ad 1.0 ml |

The pharmaceutical compositions may also contain preserving agents, solubilizing agents, stabilizing agents, wetting agents, emulsifiers, sweeteners, colorants, odorants, salts for the variation of osmotic pressure, buffers, coating agents or antioxidants. As mentioned above, they may also contain other therapeutically valuable agents.

It is a prerequisite that all adjuvants used in the manufacture of the preparations are generally recognized as safe.

Preferred forms of use are intravenous, intramuscular or oral administration, most preferred is oral administration. The dosages in which the compounds of formula (I) are administered in therapeutically effective ie the graft failure preventing amounts depend on the nature of the specific active ingredient, the age and the requirements of the patient and the mode of application. In general, dosages of about 0.001-10 mg/kg body weight per day come into consideration.

The compounds of formula (I) may also be administered in combination with anti-hypertensive drugs, hypoglycemic drugs, lipid-modulating drugs, anti-anginal drugs, anti-arrhythmic drugs, anti-thrombotic drugs, platelet aggregation inhibitory drugs, fibrinolytic drugs, anti-inflammatory drugs, anti-infective agents, immune-modulatory drugs and/or anti-proliferative drugs. Furthermore, the compounds may be administered in combination with drugs acting as receptor blockers, protein kinase inhibitors, ion channel modulators, anti-oxidants, with drugs acting on proteins such as fibrinogen and matrix metalloproteinases.

Examples of anti-hypertensive drugs are aliskiren, amlodipine, benazepril, candesartan, captopril, diltiazem, enalapril, eplenerone, eprosartan, felodipine, fosinopril, irbesartan, isradipine, lisinopril, losartan, moexipril, nicardipine, nifedipine, nisoldipine, olmesartan, perindopril, quinapril, ramipril, sildenafil, spironolactone, telmisartan, trandolapril, valsartan and verapamil;

examples of hypoglycemic drugs are insulins, repaglinide, nateglinide, glimepiridum, glibenclamidum, gliclazidum, glipizidum, glibornuridum, metformin, miglitol, acarbose, muraglitazar, pioglitazone, rosiglitazone and tesaglitazar;
examples of lipid-modulating drugs are atorvastatin, clofibrate, ezetimibe, fenofibrate, fluvastatin, gemfibrozil, lovastatin, pitavastatin, pravastatin, rosuvastatin, simvastatin;
examples of protein kinase inhibitors are imatinib, midastaurin, ruboxystaurin and staurosporine;
examples of anti-anginal drugs are acebutolol, carvedilol, glyceryl trinitrate, isosorbide mononitrate or dinitrate, labetalol, metoprolol, nadolol, nitroglycerine, pindolol, propanolol, timolol;
examples of anti-arrhythmic drugs are adenosine, amiodarone, atropine, bretylium, digoxine, disopyramide, dofetilide, flecainide, lidocaine, procainamide, propafenone, quinidine, sotalol, tocainide;
examples of anti-thrombotic drugs are acenocoumarol, argatroban, bivalirudin, cilostazol, desirudin, fondaparinux, idraparinux, lepirudin, pentoxyfylline, pheprocoumon, warfarin, ximelagatran as well as unfractionated heparin and low molecular weight heparin agents;
examples of platelet aggregation inhibitory drugs are abciximab, acetylsalicylic acid, clopidogrel, eptifibatide, ticlopidine and tirofiban;
examples of fibrinolytic agents are alfimeprase, alteplase, lanteplase, microplasmin, reteplase, streptokinase and urokinase;
examples of anti-inflammatory drugs are adalimumab, betamethasone, dexamethasone, etanercept, infliximab and prednisone;
examples of anti-infective agents aminoglycosides such as streptomycin; cephalosporins such as cefaclor, ceftriaxone and cefuroxime; macrolides such as erythromycin and azithromycin; penicillins such as amoxicillin and penicillin G; quinolones such as ciprofloxacin, norfloxacin and gatifloxacin; sulfonamides such as trimethoprim and sulfamethoxazole; tetracyclines such as minocycline and doxycyline;
examples of immune-modulatory drugs are alefacept, azathioprine, basiliximab, cyclosporine, everolismus, murmonab, mycophenolate, pimecrolismus, rapamycin, sirolsimus and tacrolismus;
examples of anti-proliferative drugs are cetuximab, docetaxel, edifoligide, gefitinib, paclitaxel and taxol.

The effectiveness of the compounds of formula (I) on the prevention of blood vessel graft failure after artery bypass graft surgery can be demonstrated using the procedure described hereafter in the example. The example illustrates the instant invention and is not meant as limiting the invention to the embodiment specifically described.

### Example

The experimental procedures to demonstrate the ability of compounds of formula (I) to prevent blood vessel graft failure after coronary artery bypass graft surgery describe the use of animal model for diet-dependent hyperlipidemia and atherosclerosis as outlined below. The model of vein graft disease consists in venous interpositions placed in the carotid arteries of hypercholesterolemic ApoA3Leiden mice. This model best reflects the complex underlying atherosclerotic stimuli leading to vessel occlusions and subsequently graft failures as observed clinically in patients.

Animals and Treatment. The murine model of vein graft disease and the illustrated experimental procedures follow basically the description in reference: Schepers et al., Journal of Vascular Surgery, 2006, volume 43, page 809-815. Male ApoE3Leiden mice on a C57/BL6 background of 14 to 20 weeks of age are used. The animals are fed a cholesterol-enriched high-fat diet (1% cholesterol and 0.05% cholate; Arie Blok, Worden, The Netherlands) starting 3 weeks prior the experiment. All mice receive water and food ad libitum. Serum cholesterol levels are determined twice i.e. 1 week before the experimental start and prior sacrificing the animals.

The mice are randomly divided into two groups. One group (n=8) receives 3-30 mg/kg of compound of formula (I) dissolved in drinking water. The daily drug dose is based on a daily water consumption of 3 ml per mouse. The other group (n=8) receives placebo in its drinking water and serves to control the experimental results. The total treatment duration is 28 days. Before surgery, mice were anesthetized with midazolam (5 mg/kg; Roche, Basel, Switzerland), medetomidine (0.5 mg/kg; Oriaon, Helsinki, Finland) and fentanyl (0.05 mg/kg; Janssen, Geel, Belgium). A venous interposition in the carotid artery is placed in each mouse. Briefly outlined, the common carotid artery is dissected free from the bifurcation at the distal end toward the proximal end. The artery is cut in the middle and cuffs are placed at the end of both sides. Subsequently, both arterial ends are everted over the cuffs and ligated with an 8-0 silk ligature. The vena cava is harvested from genetically identical donor mice and grafted between the two ends of the artery by sleeving the ends of the vein over the artery cuff and ligating them together with an 8-0 silk suture. Vigorous pulsation in the grafted vein confirms successful engraftment. At death, animals are perfused in vivo with 4% formaldehyde for 5 minutes. Vein grafts are harvested and fixed overnight in 4% formaldehyde, dehydrated and embedded in paraffin.

Quantification of vein graft thickening and immunohistochemistry. Twenty-eight days after surgery, mice are euthanized and vein grafts are harvested and embedded in paraffin. Serial cross sections of the embedded vein graft are made through the entire specimen and routinely stained with hematoxylin-phloxin-saffron (HPS). By using serial cross sections for the analysis, overestimation or underestimation of a treatment effect due to a non-equally distributed occurrence of vein graft thickening (as observed both in human vein grafts and in the murine vein graft interpositions) is prevented.

The measurement of vein graft thickening in the sampled veins is performed by using image-analysis software (Qwin; Leica, Wetzlar, Germany). Because only very few layers of cells are in the media of murine veins and because there is no morphologic border between the neointima and media, vein graft thickening, i.e., the regions between the lumen and adventitia are used to define the lesion area. For each mouse, five equally spaced perpendicular cross sections are used to determine the vessel wall thickening.

All immunohistochemistry is performed on paraffin-embedded sections of vein grafts 28 days after surgery. The cellular composition of the thickened vein grafts is visualized by using antibodies against macrophages (AIA31240; Accurate Chemical, Wesbury, USA), T cells (CD3; Sereotec, Oxford, UK) and vascular smooth muscle cells (alpha-smooth muscle actin; Amersham, Buckinghamshire, UK). The number of smooth muscle cells and macrophages is quantified by computer-assisted morphometric analysis (Qwin) and expressed as the percentage of total smooth muscle actin-positive areas or AIA-positive areas in the cross sections. The T-lymphocyte number is determined by counting CD3-positive cells in the vessel wall of six equally spaced cross sections per vein graft and divided by the vessel wall surface in these cross sections.

Statistical analysis. Before the start of each experiment, a power analysis is made to obtain statistically differentiated study arms. Data are presented as mean+/−SEM. Comparisons of morphometric data of murine vein grafts are performed with a Mann-Whitney rank sum test.

### Results

Preoperative plasma cholesterol levels do not differ between the group treated with a compound of formula (I) and the group treated with placebo. Quantification of vein graft thickening shows a dramatic thickening of the grafted vessel compared to the ungrafted vessel and a significant reduction of wall thickness in the treatment group compared with the control group. The luminal area is also increased in the grafted vessel in comparison to the ungrafted vessel and further increases in the drug treated group compared to the control group (see Table I).

The cellular composition of the thickened graft as analyzed by immunohistochemistry shows a thickened graft mainly composed of smooth muscle cells and macrophages. Furthermore, small numbers of T-lymphocytes are present in the vessel wall. The AIA-positive area that indicates the infiltration of macrophages is significantly smaller in the drug treatment group as compared to the control group. The alpha-smooth muscle actin-positive area that indicates the presence of vascular smooth muscle cells is also larger in the control group and significantly reduced in the drug treatment group. Also, the number of T-lymphocytes per square millimeter is significantly lower in the treatment group as compared to the control group.

TABLE I

| | Dose | Vessel Wall | Lumen Area | P- |
|---|---|---|---|---|
| Group | (mg/kg) | thickening (mm²) | (mm²) | value |
| Ungrafted vein | | 0.00 | 0.00 | |
| Placebo | 0 | 0.56 +/− 0.12 | 0.80 +/− 0.10 | 0.0001 |

1. Use of a compound of formula (I) 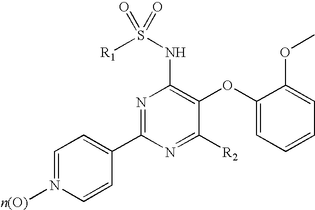
wherein
R₁ is pyridyl or thiazolyl, any of which may optionally be substituted with C₁₋₈alkyl or C₂₋₈alkenyl; and
a) R₂ is methoxy and n is zero or one; or
b) R₂ is chlorine and n is zero;
and pharmaceutically acceptable salts thereof,
for the manufacture of a medicament for the prevention of blood vessel graft failure after artery bypass graft surgery.

2. Use according to claim 1 wherein the blood vessels used for the artery bypass are of either venous or arterial phenotype such as the saphenous or cubital vein and the internal mammary (thoracic) or the gastroepiploic artery, respectively.

3. Use according to claim 1 or 2 wherein the vessel implanted by artery bypass graft surgery is implanted to a coronary artery for a coronary artery bypass graft (CABG) or a peripheral artery like the femoral artery for femoropopliteal, femorocrural bypass graft or infra-inguinal bypass surgery (IIBS).

4. Use according to one of claims 1 to 3 wherein the vessel implanted by artery bypass graft surgery is implanted to a coronary artery for a coronary artery bypass graft (CABG).

5. Use according to one of claims 1 to 4 wherein the compound of formula (I) is 5-methyl-pyridine sulfonic acid[6-methoxy-5-(2-methoxy-phenoxy)-2-pyridin-4-yl-pyrimidin-4-yl]-amide.

6. A method of prevention of blood vessel graft failure after artery bypass graft surgery that comprises the administration of an effective amount of a compound of formula (I) 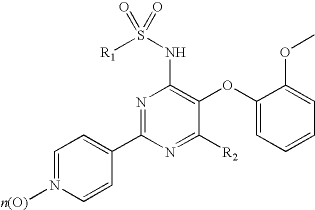
wherein
R₁ is pyridyl or thiazolyl, any of which may optionally be substituted with C₁₋₈alkyl or C₂₋₈alkenyl; and
a) R₂ is methoxy and n is zero or one; or
b) R₂ is chlorine and n is zero;
and pharmaceutically acceptable salts thereof,
to a human being or a mammalian animal.

7. A method of treatment according to claim 6 wherein the compound of formula (I) is 5-methyl-pyridine-2-sulfonic acid [6-methoxy-5-(2-methoxy-phenoxy)-2-pyridin-4-yl-pyrimidin-4-yl]-amide.

8. A pharmaceutical composition for the prevention of blood vessel graft failure after artery bypass graft surgery comprising
A) a compound of formula (I) 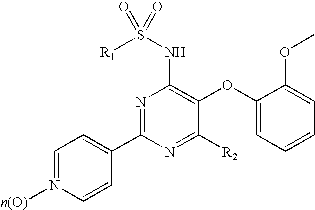
wherein
R₁ is pyridyl or thiazolyl, any of which may optionally be substituted with C₁₋₈alkyl or C₂₋₈alkenyl; and
a) R₂ is methoxy and n is zero or one; or
b) R₂ is chlorine and n is zero;
and pharmaceutically acceptable salts thereof,
and
B) one or more further compounds selected from the group comprising anti-hypertensive drugs, hypoglycemic drugs, lipid-modulating drugs, anti-anginal drugs, anti-arrhythmic drugs, anti-thrombotic drugs, platelet aggregation inhibitory drugs, fibrinolytic drugs, anti-inflammatory drugs, anti-infective agents, immunemodulatory drugs and anti-proliferative drugs and
C) an excipient.

9. A composition according to claim 8 wherein the compound of formula (I) is 5-methyl-pyridine-2-sulfonic acid[6-methoxy-5-(2-methoxy-phenoxy)-2-pyridin-4-yl-pyrimidin-4-yl]-amide.
